(19) United States
(12) Patent Application Publication
(10) Pub. No.: US 2003/0170673 A1
(43) Pub. Date: Sep. 11, 2003

(54) Identification of genes involved in restenosis and in atherosclerosis

(76) Inventors: Stephen E. Epstein, Rockville, MD (US); Sarfraz Durrani, Arlington, VA (US)

Correspondence Address: Heller Ehrman White & McAuliffe LLP, Intellectual Property Department, Suite 300, 1666 K Street, N.W., Washington, DC, 20006, US

(21) Appl. No.: 10/262,659

(22) Filed: Oct. 2, 2002

Related U.S. Application Data
(60) Provisional application No. 60/326,210, filed on Oct. 2, 2001.

(51) Int. Cl. C12Q 1/68

(52) U.S. Cl. 435/6

(57) ABSTRACT

Methods are provided for estimating the risk of developing restenosis or of atherosclerosis in an individual. Methods and compositions for treating or preventing restenosis or atherosclerosis also are provided.

### I. BACKGROUND OF THE INVENTION

Coronary artery disease is a disease that is endemic in Western society. In this disease the arteries that supply blood to the heart muscle become narrowed by deposits of fatty, fibrotic, or calcified material on the inside of the artery. The build up of these deposits is called atherosclerosis. Atherosclerosis reduces the blood flow to the heart, which starves the heart muscle of oxygen, leading to either/or angina pectoris (chest pain), myocardial infarction (heart attack), and congestive heart failure.

One common treatment to clear arteries blocked by atherosclerosis is balloon angioplasty, more formally referred to as percutaneous transluminal coronary angioplasty (PTCA). This treatment involves opening up a blocked artery by inserting and inflating a small balloon, which compresses and rearranges the blocking plaque against the arterial wall. After deflation and removal of the balloon, the arterial lumen is enlarged, thereby improving blood flow. About one million angioplasty procedures are performed each year.

In a significant number of angioplasty patients the treated artery narrows again within six months of the procedure in a process called restenosis. Restenosis begins soon after angioplasty, wherein the increased size of the vascular lumen (the open channel inside the artery) becomes gradually occluded by the proliferation of smooth muscle cells. Approximately 20 to 30% of all angioplasty patients experience restenosis to the extent that they must undergo repeated angioplasty or even coronary bypass surgery.

Restenosis has a complex pathology, triggered by the stretch-induced injury of the vessel walls during balloon inflation This stimulates smooth muscle cell migration and proliferation, and thereby leads to neointimal accumulation (which constitutes the restenotic lesion). Additional processes contributing to restenosis include inflammation and accumulation of extracellular matrix. Remodeling of the vessel wall, leading to narrowing of the vessel, is a critically important component of restenosis. However, this is totally eliminated by the implacement of a stent at the site of angioplasty, which prevents the vessel from remodeling. Stenting has become almost routine, being performed in many centers in over 70% of all angioplasty procedures. Restenosis also occurs in the arteries supplying the legs when these vessels are narrowed by atherosclerosis and are treated by angioplasty.

Currently, restenosis is diagnosed by visualizing the narrowed vessel through the injection of radioopaque dye into the vessel being examined and performing a cineangiogram (angiography). Angiography is an expensive invasive technique that requires radiation and special instruments to visualize and interpret the results. Typically, angioplasty is considered successful, not by the maintenance of the post-operative increase in the vascular lumen, but merely if the post-operative diameter of the vessel narrows less than 50% within 6-8 months of the procedure.

While several factors appear to be related to the occurrence of restenosis, including diabetes, the number of times the procedure has been performed, or the placement of a stent in the vessel, there presently are no reliable predictive indicators for the large majority of patients as to whether or not a given patient is at high risk for the development of restenosis. If a reliable risk profile were available, it would importantly influence how the patient were treated. Some patients deemed to be at very high risk for restenosis might be offered bypass surgery. Others might forego angioplasty and treated very aggressively with medical management. In still others brachytherapy (intravascular radiation) might be added to the usual angioplasty, a procedure normally reserved for patients who are now identified as being at high risk of restenosis using a rather blunt assessment—they already have had multiple episodes of restenosis. It is apparent, therefore, that new and improved methods for detecting and treating restenosis are greatly to be desired.

Finally, it is commonly appreciated that restenosis shares, with atherosclerosis, many common and overlapping processes and mechanisms. One of the key differences in these two conditions is the speed at which functionally important narrowing of the involved artery develops. Hence, restenosis can be used as an efficient model to understand many of the mechanisms responsible for atherosclerosis.

### II. SUMMARY OF THE INVENTION

It is therefore an object of this invention to provide methods for predicting the risk of the development of restenosis.

It is a further object of this invention to provide methods of treating restenosis and of reducing its recurrence.

In accomplishing these objects there is provided a method for the detection of restenosis in a mammal, comprising assaying the level of expression of at least three genes in a sample obtained from the mammal. The presence of restenosis is indicated either by increased expression of at least three, five, ten, twenty, or fifty genes in the sample, or by decreased expression of at least three, five, ten, twenty, or fifty genes in the sample. The presence of restenosis may also be indicated by the altered (raised or lowered) expression of at least three, five, ten, twenty, or fifty genes in the sample. The genes may be selected from the group of genes listed in Table 1.

The increased gene expression of a gene may be at least two fold higher, four fold higher, or ten fold higher, than a reference level. The decreased expression of a gene may be at least one-half or at least one-tenth a reference level.

The altered expression of a gene, when increased, may be at least two fold higher than a reference level of that gene and when decreased, may be one-half the level of that gene when compared to a reference level.

In each case, the said reference level may be the level in healthy (non-stenotic) vascular tissue. Alternatively, the reference level may be determined from pre-stenotic levels. The vascular tissue may be vascular arterial tissue and/or vascular venous tissue. The sample also may be blood and/or lymph.

In other embodiments, the method of assay is genetic microarray, quantitative PCR, and/or by assay of the level of protein expression in a sample. When protein expression is measured, one or more of the proteins may be soluble proteins. The level of protein expressions may be determined by ELISA.

In accordance with another object of the invention there is provided a method of inhibiting restenosis comprising administering to a patient suffering from restenosis a composition that inhibits smooth muscle cell proliferation or neointimal hyperplasia, where the composition modifies expression of at least one gene listed in Table 1. The composition may induce the expression of a gene or gene transcript that ameliorates effects of restenosis. The composition may inhibit genes that promote smooth muscle cell proliferation or neointimal hyperplasia. The composition may comprise an antisense oligonucleotide and/or an oligonucleotide that binds to mRNA to form a triplex.

In one embodiment, the composition inhibits the activity of at least one protein that promotes smooth muscle cell proliferation or neointimal hyperplasia. In another embodiment, the composition comprises an antibody that binds to a protein that promotes smooth muscle cell proliferation or neointimal hyperplasia. The composition may comprise a human antibody, and/or a soluble protein receptor. In another embodiment, the composition comprises a protein that is administered to supplement the loss of a protein down-regulated during the course of restenosis.

In a further embodiment, detection is carried out using a kit suitable for performing PCR, where the kit comprises primers specific for the amplification of DNA or RNA sequences identified by the genes in Table 1.

In accordance with another object of the invention, there is provided a method to estimate the risk of developing restenosis or of atherosclerosis in an individual, comprising detecting the presence of biologically important polymorphisms in at least three, five, ten, twenty, or fifty genes in a sample obtained from the individual. The genes may be selected from the group of genes listed in Table 1. The sample may comprise, lymph, venous or arterial blood, and/or vascular tissue of the individual. The vascular tissue may be vascular arterial tissue.

In one embodiment the polymorphisms are detected using a genetic microarray. In another embodiment the polymorphisms are detected using quantitative PCR.

In accordance with another object of the invention, there is provided a kit for carrying out any of the methods described above.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Table 1 lists the genes whose expression was detectably altered during the development of restenosis.

### DETAILED DESCRIPTION OF THE INVENTION

The invention provides new and improved methods for prediction, prevention, and treatment of restenosis and of atherosclerosis. Those genes that have altered expression levels during the healing response to acute vascular injury, and therefore during restenosis and during atherosclerosis, have been identified, and the changes in gene expression have been quantified. The relative changes in gene expression at different time points during the restenosis process have been measured, and these measurements allow additional insight into the progress and development of restenosis. Moreover, by measuring changes in gene expression, the risk of restenosis (or atherosclerosis) can be determined.

Because differential expression of genes is involved in the healing response to vascular injury, changes in the degree of expression, or in the length of time during which they are differentially expressed, lead to abnormal patterns of healing. In the context of injury to the vessel wall (either acute as in restenosis or chronic as in atherosclerosis), the excessive healing response contributes to the development of either restenosis or atherosclerosis. Changes in the degree of gene expression, or in the length of time during which the genes are differentially expressed, are caused by polymorphisms either in the gene or in the regulatory components of the gene. This invention, therefore, identifies those genes in which polymorphisms can convey susceptibility to the development of either restenosis or atherosclerosis.

The identification of genes that are involved in the healing response to acute vascular injury allows those genes having changed degree or duration of expression, caused in part by polymorphisms of the gene, to be used as targets to identify genetic abnormalities conveying altered risk of restenosis or atherosclerosis. Identification of polymorphisms associated with increased risk allows prediction of the risk for restenosis development in patients prior to the performance of the angioplasty procedure, This pre-procedure risk prediction will importantly influence how the patient is treated. Some patients deemed to be at very high risk for restenosis might be offered bypass surgery. Others might forego angioplasty and be treated aggressively with medical management. In still others brachytherapy (intravascular radiation) might be added to the usual angioplasty, a procedure normally reserved for patients who are now identified as being at high risk of restenosis using a rather blunt assessment—they already have had multiple episodes of restenosis. Accordingly, the present invention provides new and improved methods for predicting risk of restenosis.

Moreover, identification of the genes that are activated during the healing response to acute vascular injury provides new methods for preventing, ameliorating, or treating the disease by targeted inhibition of the expression of a suitable set or subset of those genes. In addition, the invention permits the monitoring of the effectiveness of restenosis treatment by measuring the changes in gene expression that occur during treatment.

Furthermore, restenosis shares, with atherosclerosis, many common and overlapping processes and mechanisms. Therefore, many of the genes differentially expressed during the healing response to acute vascular injury are the same genes differentially expressed during chronic vascular injury leading to atherosclerosis. The invention therefore also allows risk profiling of individuals for the development of atherosclerosis prior to the actual development of clinically significant atherosclerosis; i.e. prior to the development of detectable or significant narrowing of the relevant cardiac artery or peripheral arteries. This information therefore allows prophylactic intervention to prevent atherosclerosis, and prompt detection to allow delay or amelioration of the disease process.

The invention also allows the identification of genes to be analyzed for polymorphisms that predispose to atherosclerosis risk. Because different polymorphisms play a role in the development of atherosclerosis in different patients, the invention allows identification of specific abnormalities that may be characteristic to a specific patient. The invention therefore allows for greater specificity of treatment. A regime that may be efficacious in one patient with a specific polymorphism profile may not be effective in a second patient with a different polymorphism profile. Such a profiling also allows treatment to be individualized so that unnecessary side effects of a treatment strategy that would not be effective for a specific patient can be avoided.

Specifically, approximately two hundred genes are identified whose expression changes during the course of the healing response to acute vascular injury—the intrinsic process leading to restenosis.

Since the differential expression of these genes is involved in the healing response to vascular injury, changes in the degree of expression, or in the length of time during which they are differentially expressed, could lead to abnormal patterns of healing. Analogous to a keloid scar, in which a genetic precondition leads to excessive fibrous tissue developing on the skin in response to cutaneous injury, in the context of injury to the vessel wall (either acute as in restenosis or chronic as in atherosclerosis), the excessive healing response can contribute to the development of restenosis.

Changes in the degree of gene expression, or in the length of time during which the genes are differentially expressed, can be caused by polymorphisms in the gene or in the regulatory components of the gene. Such polymorphisms, conveying an increased risk of disease development, have already been identified for several genes associated with several diseases. This invention, therefore, identifies those genes in which polymorphisms can convey susceptibility to the development of restenosis. Subsequent reference, therefore, to prediction of restenosis (or atherosclerosis-see below), relate to polymorphisms of the genes identified by this invention, or of their regulatory units.

Restenosis may be predicted by identifying polymorphisms of at least three genes whose expression is up-regulated during the healing response to acute vascular injury. Identification of polymorphisms of at least three of those genes down-regulated during the healing response to acute vascular injury is predictive of restenosis. In addition, identification of polymorphisms of at least three genes, some of which are up-regulated and some of which are down-regulated, is predictive of restenosis. Further, the expression of some genes is altered during the course of the healing response to acute vascular injury.

The change in expression of certain of the identified genes is predictive, not just of the risk for restenosis itself, but is diagnostic of the stage of development of the disease. By identifying almost 200 genes whose expression changes during the healing response to acute vascular injury and therefore during the development of restenosis, the inventors recognize that analysis of greater numbers of polymorphisms of those genes leads to a greater ability to predict the development of restenosis, to determine the probability of its development, and to predict its ultimate severity. In view of the importance that the identified genes may play in the etiology of restenosis, an ability to manipulate the expression of those genes may be efficacious in the treatment of restenosis. Methods to treat restenosis may include gene therapy to increase the expression of genes down-regulated during the disease. Treatment may also include methods to decrease the expression of genes up-regulated during restenosis. Treatment to decrease gene expression may include, but is not limited to, the expression of anti-sense mRNA, triplex formation or inhibition by co-expression.

Identification of genes involved in the development of restenosis also makes possible an identification of proteins that may effect the development of restenosis. Identification of such proteins makes possible the use of methods to affect their expression or alter their metabolism. Methods to alter the effect of expressed proteins include, but are not limited to, the use of specific antibodies or antibody fragments that bind the identified proteins, specific receptors that bind the identified protein, or other ligands or small molecules that inhibit the identified protein from affecting its physiological target and exerting its metabolic and biologic effects. In addition, those proteins that are down-regulated during the course of restenosis may be supplemented exogenously to ameliorate their decreased synthesis.

The identification of genes involved in the development of restenosis makes possible the prophylactic use of methods to affect gene expression or protein function, and such methods may be used to treat individuals at risk for the development of restenosis.

Different polymorphisms may play a role in the development of restenosis in different patients. Accordingly, the present invention makes possible an identification of specific abnormalities that are characteristic of a specific patient, which allows for greater specificity of treatment. A regime that may be efficacious in one patient with a specific polymorphism profile may not be effective in a second patient with a different polymorphism profile. Such a profiling also allows treatment to be individualized so that unnecessary side effects of a treatment strategy that would not be effective for a specific patient can be avoided.

Finally, restenosis shares, with atherosclerosis, many common and overlapping processes and mechanisms. One of the key differences in these two conditions is the speed at which functionally important narrowing of the involved artery develops. Hence, restenosis can be used as an efficient model to understand many of the mechanisms responsible for atherosclerosis. Accordingly, each of the methods disclosed herein may be used to predict the occurrence of atherosclerosis as well as restenosis. Similarly, the methods of treatment disclosed herein may be used to treat, prevent, and/or ameliorate the symptoms of atherosclerosis as well as restenosis

Elucidation of Changes in Gene Expression in Restenosis

The present inventors have identified the genes that undergo changes in expression during the healing response to acute vascular injury, and therefore during the process of restenosis. Those genes are listed in Table 1. The inventors have carried out this analysis using nucleic acid array analysis of rat cardiac tissue as described in more detail below.

The rat is a widely accepted model for the human for vascular studies, and results obtained in the rat are considered highly predictive of results in humans. Accordingly, it is expected that the changes in gene expression in humans during the healing response to acute vascular injury will be similar to or essentially the same as those observed in the rat. Exaggerated changes in the degree of expression in these genes, or in the length of time during which the genes are differentially expressed, will predispose to restenosis. Such exaggerated changes are usually caused by polymorphisms in the gene or in the regulatory components of the gene, and therefore the rat genes identified as being differentially regulated during the healing response to acute vascular injury will be homologous to the human genes in which such polymorphisms will be found to convey susceptibility to restenosis. Moreover, both rat and human homologues are known for each of the genes described in Table 1, demonstrating further that the results obtained in the rat studies will be highly predictive of results obtained in humans.

Because restenosis shares many of the processes and mechanisms as atherosclerosis, and since both result from vascular injury, then the genes identified in the rat model of the healing response to acute vascular injury will also be the genes whose abnormal expression will predispose to atherosclerosis. The specific abnormalities are determined by identifying polymorphisms of these genes that are associated with atherosclerosis. Such genes also serve as the target for therapeutic interventions—those genes upregulated during the healing response to acute vascular injury can be targeted by therapy designed to decrease gene expression or function of the proteins encoded by these genes; those genes down-regulated during the healing response to acute vascular injury can be targeted by therapy designed to increase gene expression or function of the proteins encoded by these genes.

Changes in gene expression in the rat carotid artery during experimentally induced acute vascular injury have been studied, a model commonly accepted as a reasonable animal model simulating restenosis as it occurs in humans. Sample and control rat carotid artery tissues were obtained, RNA was prepared from the tissues, labeled cRNA generated from it and analyzed using an Affymetrix GeneChip® Rat Genome U34A Set. Sample and control tissues were compared and those genes that experienced significant changes in gene expression were identified. For the purposes of this study, a two fold increase or decrease in gene expression was deemed significant, although the skilled worker will recognize that under certain circumstances smaller changes in gene expression may also be significant. Corresponding human genes for each of the genes determined to have a significant change in expression were identified.

Now that an essentially complete set of genes that undergo changes in expression in the healing response to acute vascular injury has been identified, it is possible to predict the risk of restenosis and/or atherosclerosis developing by studying the changes of a smaller subset of those genes. Thus, although about 200 genes have been shown to have altered expression in restenosis, it is possible to reliably predict the risk of restenosis by analyzing a subset of these genes that contains as few as three members. In other embodiments, at least five, ten, fifteen, twenty or fifty genes may be studied or, if desired, all or most of the genes listed in Table 1 can be studied. Moreover, these genes can also be analyzed for polymophisms associated with restenosis and atherosclerosis. All of the genes can be analyzed intially, but reliable predictions can be made by analyzing a subset of these genes that contains as few as three members. In other embodiments, at least five, ten, fifteen, twenty or fifty genes may be studied or, if desired, all or most of the genes listed in Table 1 can be studied, for example, using sequencing, short tandem repeat association studies, single nucleotide polymorphism association studies, etc. In each case, however, it generally is more convenient to study gene expression or polymorphisms in a smaller subset of the genes.

By measuring changes in expression of a set of genes (or by identification of polymorphisms influencing expression of sets of genes), rather than of a single gene, the present invention provides increased statistical confidence that the changes observed are predictive of the risk of developing restenosis or atherosclerosis—ie, provides reliable risk profiling of an individual. Thus, a change in expression of a single gene, or a single gene polymorphism, may not increase susceptibility to disease sufficiently to cross the threshold for disease development. On the other hand, coordinated changes in expression of multiple specified genes, due the presence of multiple polymorphisms, is much more likely increase the risk of restenosis or of atherosclerosis. This is analogous to the situation of an individual have only one risk factor predisposing to atherosclerosis (elevated cholesterol). Risk is increased markedly as the number of risk factors increase (elevated cholesterol plus hypertension, obesity, smoking, diabetes, etc).

By assaying gene expression and/or the presence of polymorphisms that influence expression of these genes it is possible to predict the risk of restenosis development prior to performing the angioplasty procedure, and predict the risk of atherosclerosis development prior to the development of clinically detectable atherosclerosis. Such early prediction provides the clinician with opportunities to slow or halt the restenosis or atherosclerosis Moreover, the invention provides new compositions that can be used to inhibit, slow, or prevent restenosis and atherosclerosis.

Dysregulation of Multiple Genes that Increase Susceptibility to Restenosis or to Atherosclerosis

Gene polymorphisms that lead to biologically important exaggerated changes in the expression of genes that are differentially expressed during the course of the healing response to acute vascular injury, and which thereby predispose to restenosis or atherosclerosis, can be measured directly in patient samples. These samples comprise DNA that is most conveniently obtained from peripheral blood. The present inventors used nucleic acid array methods to identify the complete set of genes that exhibit significantly changed expression during the course of the healing response to acute vascular injury. However, other methods for measuring changes in gene expression are well known in the art. For example, levels of proteins can be measured in tissue sample isolates using quantitative immunoassays such as the ELISA. Kits for measuring levels of many proteins using ELISA methods are commercially available from suppliers such as R&D Systems (Minneapolis, Minn.) and ELISA methods also can be developed using well known techniques. See for example Antibodies: A Laboratory Manual (Harlow and Lane Eds. Cold Spring Harbor Press). Antibodies for use in such ELISA methods either are commercially available or may be prepared using well known methods.

Other methods of quantitative analysis of multiple proteins include, for example, proteomics technologies such as isotope coded affinity tag reagents, MALDI TOF/TOF tandem mass spectrometry, and 2D-gel/mass spectrometry technologies. These technologies are commercially available from, for example, Large Scale Proteomics Inc. (Germantown Md.) and Oxford Glycosystems (Oxford UK).

Alternatively, quantitative mRNA amplification methods, such as quantitative RT-PCR, can be used to measure changes in gene expression at the message level. Systems for carrying out these methods also are commercially available, for example the TaqMan system (Roche Molecular System, Alameda, Calif.) and the Light Cycler system (Roche Diagnostics, Indianapolis, Ind.). Methods for devising appropriate primers for use in RT-PCR and related methods are well known in the art. In particular, a number of software packages are commercially available for devising PCR primer sequences.

Nucleic acid arrays offer are a particularly attractive method for studying the expression of multiple genes. In particular, arrays provide a method of simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). See also U.S. Pat. Nos. 5,445,934, 5,700,637, 6,080,585, 6,261,776 which are hereby incorporated by reference in their entirety.

Changes in the degree of gene expression, or in the length of time during which the genes are differentially expressed, can be caused by polymorphisms in the gene or in the regulatory components of the gene. Such polymorphisms, conveying an increased risk of disease development, have already been identified for genes associated with several diseases. The present invention, therefore, identifies those genes in which polymorphisms can convey susceptibility to the development of restenosis or atherosclerosis.

To study a set of genes having altered expression in restenosis using nucleic acid arrays, samples of total RNA or mRNA are obtained from cardiac tissue, and analyzed using methods that are well known in the art. Thus, for example, samples of suitable cardiac tissue, such as carotid artery, can be obtained by biopsy. Total RNA can be obtained using commercially available kits, such as Triazol reagent (Invitrogen, Carlsbad, Calif.) and mRNA can be obtained from this sample by chromatography on oligo(dT) cellulose. The RNA is reverse transcribed and the resulting cDNA subjected to an amplification step. In one embodiment, the amplification is a linear RNA amplification method such as that described in U.S. Pat. Nos. 5,716,785 and 5,891,636, which are hereby incorporated by reference in their entirety. Detailed instructions for preparing amplified RNA are available, for example, in the manufacturer's directions for preparing samples for assay using the Affymetrix GeneChip system.

Once suitable nucleic acid samples have been obtained, the gene expression profiles are determined using the nucleic acid arrays according to the manufacturer's instructions. For every gene probe on the array this provides a quantitative gene expression level in the sample. The expression level for each gene can then be compared to a baseline value to determine whether expression has been altered. Thus, the gene expression level of genes in tissue under study can be compared to reference levels of those genes in healthy tissue where restenosis is not occurring. Preferably, those reference levels are obtained from the same, although it is possible to use reference levels from different subjects. In such cases it is preferred to use reference levels from subjects that resemble the test subject as closely as possible, for example in demographic criteria such as age, gender, ethnicity, etc.

Although it is possible to measure absolute gene expression levels, it often is more convenient to measure relative gene expression levels. Thus, levels of expression of a particular gene on the array are compared to a reference gene on the same array whose expression is known to be unaffected in restenosis, for example, a gene not shown in Table 1. This provides an internal control mechanism for the array and reduces any differences in results that are due to variability in the array, assay conditions, etc.

In each case, the level of gene expression is compared to a suitable baseline level of expression. The baseline level of expression can be the level found in healthy vascular tissue, the level assayed prior to angioplasty, a global concentration assayed from a pool of healthy individuals or some other objective baseline.

Methods for identifying polymorphisms in genes are well known in the art. See, for example, U.S. Pat. Nos. 6,235,480 and 6,268,146, which are hereby incorporated by reference. Once polymorphisms are identified, methods for detecting specific polymorphisms in a gene using nucleic acid arrays are also well known in the art

Thus, in one embodiment, the invention provides methods where the expression of at least three genes selected from the genes shown in Table 1 are assayed. The genes can be selected in combinations such that (i) increased expression of all three genes indicates restenosis; (ii) decreased expression of all three genes indicates restenosis; (iii) decreased expression of some gene(s) combined with increased expression of the remaining selected genes indicates restenosis, or (iv) decreased expression of some genes and the increased expression of other genes at the beginning or shortly after angioplasty followed by an increase in the expression of those down-regulated genes and a decrease in the expression of genes initially up-regulated is indicative of the development of restenosis. In other embodiments of the invention the expression of at least five genes or at least about five genes is assayed to determine the development of restenosis. In yet further embodiments the number of genes assayed is ten. In yet other embodiments the number of genes assayed is 20 or at least about 20. In still yet other embodiments the number of genes assayed is 50 or at least about 50. Regardless of the number of genes in the subset of analyzed genes, the expression profile satisfies the criteria to diagnose the disease set out above when (i) the expression of some genes is increased throughout the course of the disease; (ii) the expression of some genes is decreased throughout the course of the disease; (iii) expression of some of the genes are increased while others are decreased, or (iv) the expression of some genes is altered during the development of the disease.

The invention also provides methods where the presence of at least three gene polymorphisms, selected from the genes shown in Table 1, are assayed. The aggregate number of polymorphisms can then provide an estimate of risk of restenosis or atherosclerosis. The more biologically significant polymorphisms are present, the greater the risk. As more polymorphisms of the genes listed in Table 1 are identified, even more powerful risk profiling will be possible. Thus, in other embodiments of the invention the expression of at least five genes or at least about five genes is assayed to determine the risk of developing restenosis or atherosclerosis. In yet further embodiments the number of genes assayed is ten. In yet other embodiments the number of genes assayed is 20 or at least about 20. In still yet other embodiments the number of genes assayed is 50 or at least about 50.

Regardless of the number of genes in the subset of analyzed genes, the polymorphism profile satisfies the criteria to determine the risk of developing restenosis or atherosclerosis set out above when the aggregate number of polymorphisms in the genes listed in Table 1 that exaggerate gene expression in biologically significant ways and that thereby predispose to the development of restenosis or atherosclerosis. The skilled artisan will recognize that, due to the heterogeneous nature of restenosis and of atherosclerosis, not all individuals with restenosis or atherosclerosis will exhibit altered expression of every last one of the genes listed in Table 1. Thus, it is possible that one, a few, or many genes will not exhibit significantly altered expression (and therefore will contain no biologically important polymorphisms), and that different individuals will exhibit different combinations of polymorphisms; yet, the coordinated changes induced by the polymorphisms in the expression of the totality of genes are highly predictive of the presence of development of restenosis and of atherosclerosis.

In general, where the expression of only a relatively small number of genes is studied, changes in expression in most or all of the genes must be observed to provide a reliable diagnosis of restenosis or atherosclerosis. For example, where only three genes are measured, all three genes must show relevant changes in expression to permit a reliable diagnosis of restenosis or atherosclerosis. Where five genes are studied, changes in at least four genes typically will provide a reliable diagnosis. Where ten genes are measured, a reliable diagnosis is obtained where changes in at least seven genes are observed. Where more than 10 genes are measured, changes in 90%, 80%, 70%, 60% or 50% of the measured genes are predictive of restenosis or atherosclerosis. As these percentages decrease, the reliability of the diagnosis also decreases, but the skilled worker will recognize that when a coordinated change in expression of 20 or 30 genes of the genes listed in Table 1 is observed this is highly predictive of the presence of restenosis. In general, as the number of genes increases, it is possible to provide a reliable diagnosis by observing coordinated changes in expression in a relatively smaller subset of the genes studied.

In general, where biologically important polymorphisms (leading to a predisposition to restenosis or of atherosclerosis) of only a relatively small number of genes is studied, polymorphisms in most or all of the genes must be observed to provide a reliable estimate of risk of developing restenosis or of atherosclerosis. For example, where only three genes are measured, all three genes must show relevant polymorphisms to permit a reliable estimate of risk of developing restenosis or of atherosclerosis. Where five relevant polymorphisms or ten polymorphisms are identified, the greater the number of such polymorphisms an individual has the greater the estimated risk of developing restenosis or of atherosclerosis. The skilled worker will recognize that when a coordinated change in expression of 20 or 30 genes of the genes listed in Table 1 is observed (as a result biologically important polymorphisms) this is highly predictive of the risk of developing restenosis or of atherosclerosis.

Tissues Sampled to Determine Altered Gene Expression and the Presence of Polymorphisms that Cause Biologically Important Alterations in Relevant Gene Expression

Although any sample containing nucleic acid would be appropriate for this purpose, the simplest tissue to sample is peripheral venous or arterial blood. However, tissue may be used, such as vascular tissue, in particular arterial vascular tissue or venous vascular tissue.

Significance of Altered Gene Expression

The terms increased expression, decreased expression or altered expression mean at least a two fold difference or at least about a two fold difference in the expression of the identified gene when compared to the expression of that gene in a control or non-restenotic animal. The change in gene expression may be at least four fold higher or at least about four fold higher than the reference level. In yet other embodiments the change in gene expression is at least ten fold higher or at least about ten fold higher than the reference level. Because some of the genes identified are down-regulated the term decreased expression means those genes that have at least a two-fold decrease or at least a about two-fold decrease in expression compared to control values. In other embodiments the term decreased expression means those genes that have at least a ten-fold decrease or at least about a ten-fold decrease in expression when compared to reference values.

Determination of Reference Level

The reference level used in the methods of the present invention is the level of gene expression in relatively healthy vascular tissue. This may mean the level of gene expression in pre-stenotic tissue, or it may mean the level of gene expression prior to angioplasty. The reference level may be determined from global values assayed from healthy individuals.

Methods of Studying Gene Expression and Polymorphisms of the Genes Listed in Table 1

Gene expression may be studied at the nucleic acid (RNA) level or the protein level. While each cell nucleus carries a complete set of genes only those genes expressed in each cell are transcribed into mRNA which is then translated into proteins. Consequently, gene expression is tissue or even cell specific. Generally, it is thought that the greater the number of RNA molecules transcribed the greater the number of protein molecules translated from them and, accordingly, the results obtained using RNA or protein analysis should be the same, at least in terms of relative changes in levels of gene expression. An analysis of gene expression may therefore be directed at the quantity of a particular mRNA transcript or the amount of protein translated from it.

Polymorphisms can be identified by several methods including sequencing, short tandem repeat association studies, single nucleotide polymorphism association studies, etc. These methods are well-known in the art.

Gene expression can also be studied at the protein level. While each cell nucleus carries a complete set of genes only those genes expressed in each cell are transcribed into mRNA which is then translated into proteins. Consequently, gene expression is tissue or even cell specific. Generally, it is thought that the greater the number of RNA molecules transcribed the greater the number of protein molecules translated from them and, accordingly, the results obtained using protein analysis should be the same, at least in terms of relative changes in levels of gene expression. An analysis of gene expression may therefore be directed at the quantity of a particular mRNA transcript or the amount of protein translated from it. However, although gene polymorphisms are detected reliably with tissue derived from any source, including peripheral blood, assay of the mRNA or protein encoded by any of the genes listed in Table 1 to determine relevant changes in the level of gene expression is critically dependent on tissue sampled. While some idea of altered gene expression occurring at the site of developing restenosis or of atherosclerosis can be obtained from sampling and testing peripheral blood, much more reliable estimates of altered gene expression would be obtained from sampling the actually artery developing restenosis or of atherosclerosis.

RNA Expression

Methods of isolating RNA from tissue are well known in the art. See, for example, Sambrook et al. Molecular Cloning: A Laboratory Manual (Third Edition) Cold Spring Harbor Press, 2001. Commercial reagents also are available for isolating RNA.

Briefly, for example, cells or tissue are lysed and the lysed cells centrifuged to remove the nuclear pellet. The supernatant is then recovered and the nucleic acid extracted using phenol/chloroform extraction followed by ethanol precipitation. This provides total RNA, which can be quantified by measurement of optical density at 260-280 nM.

mRNA can be isolated from total RNA by exploiting the “PolyA” tail of mRNA by use of several commercially available kits. QIAGEN mRNA Midi kit (Cat. No. 70042); Promega PolyATtract® mRNA Isolation Systems (Cat. No. Z5200). The QIAGEN kit provides a spin column using Oligotex Resin designed for the isolation of poly A mRNA and yields essentially pure mRNA from total RNA within 30 minutes. The Promega system uses a biotinylated oligo dT probe to hybridize to the mRNA poly A tail and requires about 45 minutes to isolate pure mRNA.

mRNA can also be isolated by using the cesium chloride cushion gradient method. Briefly the flash frozen tissue if homogenized in Guanethedium isothiocyanate, layered over a cushion of cesium chloride and ultracentrifuged for 24 hours to obtain the total RNA.

Genetic Microarray Analysis

Microarray technology is an extremely powerful method for assaying the expression of multiple genes in a single sample of mRNA. For example, Gene Chip® technology commercially available from Affymetrix Inc. (Santa Clara, Calif.) uses a chip that is that is plated with probes for over thousands of known genes and expressed sequence tags (ESTs). Biotinylated cRNA (linearly amplified RNA) is prepared and hybridized to the probes on the chip. Complementary sequences are then visualized and the intensity of the signal is commensurate with the number of copies of mRNA expressed by the gene.

Quantitative PCR

Quantitative PCR (qPCR) employs the co-amplification of a target sequence with serial dilutions of a reference template. By interpolating the product of the target amplification with that a curve derived from the reference dilutions an estimate of the concentration of the target sequence may be made. Quantitative reverse transcription PCR (RTPCR) may be carried out on mRNA using kits and methods that are commercially available from, for example, Applied BioSystems (Foster City, Calif.) and Stratagene (La Jolla, Calif.) See also Kochanowsi, Quantitative PCR Protocols” Humana Press, 1999. For example, total RNA may be reverse transcribed using random hexamers and the TaqMan Reverse Transcription Reagents Kit (Perkin Elmer) following the manufacturer's protocols. The cDNA is amplified using TaqMan PCR master mix containing AmpErase UNG dNTP, AmpliTaq Gold, primers and TaqMan probe according to the manufacture's protocols. The TaqMan probe is target-gene sequence specific and is labeled with a fluorescent reporter (FAM) at the 5′ end and a quencher (e.g. TAMRA) at the 3′ end. Standard curves for both endogenous control and the target gene may be constructed and the comparison of the ration of CT (threshold cycle number) of target gene to control in treated and untreated cells is determined. This technique has been widely used to characterize gene expression.

Protein Expression

Gene expression may also be studied at the protein level. Target tissue is first isolated and then total protein is extracted by well known methods. Quantitative analysis is achieved, for example, using ELISA methods employing a pair of antibodies specific to the target protein.

A subset of the proteins listed in Table 1 are soluble or secreted. In such instances the proteins may be found in the blood, plasma or lymph and an analysis of those proteins may be afforded by any of those methods described for the analysis of proteins in such tissues. This provides a minimally invasive means of obtaining patient samples for estimate of risk of developing restenosis or of atherosclerosis. Methods for identifying secreted proteins are known in the art.

Treatment of Restenosis

The identification of the set of genes having altered expression during the healing response to acute vascular injury, provides new opportunities to treat restenosis or atherosclerosis. Identification of genes up-regulated during the healing response to acute vascular injury affords the ability to use methods to negatively affect their transcription or translation. Similarly, the identification of genes that are down-regulated during the healing response to acute vascular injury affords the ability to positively affect their expression. Finally, the determination of the proteins encoded by these genes allows for the use of appropriate methods to ameliorate or potentiate the protein activities, which thereby could influence the development of restenosis or atherosclerosis.

Methods of Enhancing Gene Expression

For genes that exhibit decreased expression during the the healing response to acute vascular injury, it is possible to ameliorate or prevent restenosis or atherosclerosis by enhancing expression of one or more of these genes. Gene transcription may be deliberately modified in a number of ways. For example, exogenous copies of a gene may be inserted into the genome of cells in vascular tissue by genomic transduction via homologous recombination. While expression by genomic transduction is relatively stable it also is of low efficiency. An alternative method is transient transduction where the gene is inserted within a vector allowing for its transcription independent of the genomic allele making use of a vector specific promoter. While transient transduction generally has a higher expression the gene is maintained for a much shorter period of time, although use of episomal vector containing a eukaryotic origin of transcription provides for greater persistence of the vector. Yet another method is transfection with naked DNA. However, this method generally results in very low expression and the DNA appears to be rapidly degraded.

Methods of Inhibiting of Gene Expression

The present invention also affords an ability to negatively affect the expression of genes that are up-regulated during the healing response to acute vascular injury. Methods for down regulating genes are well known. It has been shown that antisense RNA introduced into a cell will bind to a complementary mRNA and thus inhibit the translation of that molecule. In a similar manner, antisense single stranded cDNA may be introduced into a cell with the same result. Further, co-suppression of genes by homologous transgenes may be effected because the ectopically integrated sequences impair the expression of the endogenous genes (Cogoni et al. Antonie van Leeuwenhoek, 1994; 65(3):205-9), and may also result in the transcription of antisense RNA (Hamada and Spanu, Mol. Gen Genet 1998). Methods of using short interfering RNA (RNAi) to specifically inhibit gene expression in eukaryotic cells have recently been described. See Tuschl et al., Nature 411:494-498 (2001).

In addition, stable triple-helical structures can be formed by bonding of oligodeoxyribonucleotides (ODNs) to polypurine tracts of double stranded DNA. (See, for example, Rininsland, Proc. Nat'l Acad. Sci. USA 94:5854-5859 (1997). Triplex formation can inhibit DNA replication by inhibition of transcription of elongation and is a very stable molecule.

Methods to Inhibit the Activity of Specific Proteins

When a specific protein has been implicated in the restenotic or atherosclerotic pathway its activity can be altered by several methods. First, specific antibodies may be used to bind the protein thereby blocking its activity. Such antibodies may be obtained through the use of conventional hybridoma technology or may be isolated from libraries commercially available from Dyax (Cambridge, Mass.), MorphoSys (Martinsried, Germany), Biosite (San Diego, Calif.) and Cambridge Antibody Technology (Cambridge, UK). In addition, proteins usually exert their cellular effects by ligating to cellular receptors. Identification of the receptors to which proteins, which are implicated by the current invention as contributing to restenosis or atherosclerosis, bind will allow the design of specific ligand antagonists that block pathways mediating the effects leading to the development of restenosis or atherosclerosis.

The identification of genes that are down regulated during the the healing response to acute vascular injury leads to the ability to identify their protein products. Down-regulated proteins may then be supplemented, thereby ameliorating the effect of their decreased synthesis.

The methods of the present invention may be used prophylactically to prevent the development of restenosis or atherosclerosis in at risk individuals.

The present invention also provides kits having chips containing the DNA of the biologically important polymorphisms for the genes identified in Table 1. Such chips permit the rapid detection of the polymorphisms, providing a convenient means for the rapid detection of those individuals at high or at low risk of developing restenosis or of atherosclerosis. The detection of specific polymorphisms in specific patients will allow highly specific and individualized treatment strategies to be devised for each patient to prevent or attenuate restenosis and or atherosclerosis.

The present invention, thus generally described, will be understood more readily by reference to the following example, which is provided by way of illustration and is not intended to be limiting of the present invention.

### EXAMPLE

### Microarray Analysis of the Restenotic Carotid Artery

Isolation of RNA

Rats were divided into two groups. One group was treated with carotid angioplasty and the control group was treated by sham surgery. Rat carotids after surgery and sham surgery were collected and flash frozen. Pooled carotids (30-50 mg) were crushed into powder using a mortar and pestle (collected with liquid nitrogen) and then homogenized in 2.5 ml of guanidinium isothiocyanate. Total RNA was extracted using ultracentrifugation on cesium chloride cushion gradient for 24 hours at 4° C. See Sambrook et al supra.

Target Preparation and DNA Microarray Hybridizations

For the first strand cDNA synthesis reaction, 5.0-8.0 μg of total RNA was incubated at 70° C. for 10 minutes with T7-(dT) 24 primer, then placed on ice. For the temperature adjustment step, 5×first stand cDNA buffer, 0.1 M DTT, and 10 mM dNTP mix was added and the reaction incubated for 1 hour at 42° C. SSII reverse transcriptase was added, and the reaction incubated for 1 hour at 42° C. With the first strand synthesis completed, 5×second strand reaction buffer, 10 mM dATP, dCTP, dGTP, dTTP, DNA Ligase, DNA Polymerase I, and RNaseH were added to the reaction tube. Samples were then incubated at 16°. Following the addition of 0.5M EDTA, cDNA was cleaned using phase lock gels-phenol/chloroform extraction, followed by ethanol precipitation.

Synthesis of Biotin-Labeled cRNA (In Vitro Transcription)

The synthesis of biotin-labeled cRNA was completed using the ENZO BioArray RNA transcript labeling kit from (ENZO Biochem, Inc., New York, N.Y.) according to the manufacturers protocol. To set up the reaction 1 μg of cDNA, 10×HY reaction buffer, 10×Biotin labeled ribonucleotides, 10×DTT, 10×RNase inhibitor mix and 20×T7 RNA polymerase were incubated at 37° C. for 4-5 hours. RNeasy spin columns from QIAGEN were used to purify the labeled RNA, followed by ethanol precipitation and quantification.

Fragmentation of cRNA for Target Preparation

5×fragmentation buffer (200 mM Tris-acetate, pH 8.1, 500 mM KOAc, 150 mM Mg)Ac) was added to the cRNA. Samples were incubated at 94° C. for 35 minutes, then placed on ice. Fragmented cRNA was stored at −70° C.

Target Hybridization

Hybridization cocktail was prepared as follows: fragmented cRNA (15 μg adjusted), control oligonucleotide B2 (Affymetrix), 20×eukaryotic hybridization controls (Affymetrix), herring sperm DNA, acetylated BSA, and 2×hybridization buffer (Affymetrix) were combined, and heated to 99° C. for five minutes. Hybridization cocktail was then centrifuged at maximum speed for five minutes to remove any insoluble materials from the mixture. Following centrifugation, cocktail was heated at 45° C. for five minutes. The clarified hybridization cocktail was then added to the Affymetrix U34A probe array cartridge that had been pre-wet with 1×hybridization buffer. The probe array was then placed in a 45° C. rotisserie box oven set at 60 rpm and hybridized for 16 hours.

Washing, Staining and Scanning Probe Arrays

The GeneChip® Fluidics Station 400 was used to wash and stain the array. This instrument was run using GeneChip® software. Briefly, arrays were washed for 10 cycles with non-stringent wash buffer at 25° C., followed by 4 cycles of washing with stringent wash buffer at 50° C. The array was then stained for 10 minutes with Phycoerythrin-streptavidin at 25° C. The array was then washed for 10 cycles with non-stringent wash buffer at 25° C. The probe array was the stained again with phycoerythrin-streptavidin for 10 minutes at 25° C., and then washed for 15 cycles with non-stringent wash buffer at 30° C. Hybridization signals are detected by placing the probe array in an HP Gene Array™ Scanner, which operated using GeneChip® software.

Data Analysis

Data analysis was performed using GeneChip® software (version 3.3) using the manufacturer's instructions. Lockhart, D. J. et al., Nat. Biotechnol. 14:1675-80 (1996). Briefly, each gene was represented and queried by 1-3 probe sets on the chip. Each probe set comprises 16 perfect match (PM) and 16 mismatch (MM) 25 nucleotide base probes, The mismatch has a single base change in the middle of the 25 base pair probe. The hybridization signal from the PM and the MM probes were compared and this allowed for a measure of signal intensity that is specific and eliminated the nonspecific cross hybridization from the data of the two control chips. Intensity differences as well as ratios of intensity of each probe pair are used to make a “present” or “absent” call. The controls were used as baseline and the experimental GeneChip® assay values compared to the base line to derive four matrixes which were used to determine the difference calls that indicate whether the transcription level of a particular gene is changed.

Iterative comparisons were performed using a spreadsheet analysis (Microsoft Excel). Each experimental data set at a particular time point (n=2) and the difference in expression between the controls and experimental was determined for each gene. Genes with a consistent difference call across all four pairwise comparisons were extracted for further analysis.

GeneSpring® Analysis

The data from each GeneChip® assay was fed into the GeneSpring® software and clustering of genes based on their temporal expression profile was analyzed. Correlation coefficients of 0.97 or greater were taken as a cutoff to create gene-clusters with significant expression homology.

This application claims priority from U.S. application Ser. No. 60/326,210, the specification of which is incorporated by reference in its entirety.

RAT GENES IN RESTENOSIS: 3 HOUR DOWN REGULATED

| | A | B | C | D | |
|---|---|---|---|---|---|
| 1 | Rat Accession # | Locus link | DESCRIPTION | Human locus | E |
| 2 | | | | | |
| 3 | AF022136_at | GJA5 | GJA5 gap junction protein, alpha | LocusID: 2702 | |
| | | | 40kD (connexin 40) | | |
| 4 | J00738_s_at | CALD1 | CALD1: caldesmon 1 | LocusID: 800 | |
| 5 | | | | | |
| 6 | K01934mRNA#2_at | THRSP | THRSP: thyroid hormone | LocusID: 7069 | |
| | | | responsive SPOT14 (rat) | | |
| | | | homolog | | |
| 7 | rc_AI237731_s at | LPL | LPL: lipoprotein lipase | LocusID: 4023 | |
| 8 | L03294_at | LPL | LPL: lipoprotein lipase | LocusID: 4023 | |
| 9 | L03294_g_at | LPL | LPL: lipoprotein lipase | LocusID: 4023 | |
| 10 | L46791_at | CES3 | CES3 carboxylesterase 3 (brain) | LocusID: 23491 | |
| 11 | | | | | |
| 12 | A04674cds_s_at | 7350 | UCP1: uncoupling protein 1 | LocusID: 7350 | |
| | | | (mitochondrial, proton carrier) | | |
| 13 | X03894_at | | UCP: BROWN ADIPOSE | LocusID: 7350 | |
| | | | TISSUE UNCOUPLING | 4q31 | |
| | | | PROTEIN | | |
| | | | THERMOGENIN | | |
| 14 | | | | | |
| 15 | M22400_at | GPG3 | GPC3: glypican 3 | LocusID: 2719 | |
| 16 | U66470_at | CGR11 | CGR11: cell growth regulatory | LocusID: 10669 | |
| | | | with EF-hand domain | | |
| 17 | U95178_s_at | 1601 | DAB2: disabled (Drosophila) | LocusID: 1601 | |
| | | | homolog 2 (mitogen-responsive | | |
| | | | phosphoprotein) | | |
| 18 | J03179_at | DBP | DBP: D site of albumin promoter | LocusID: 1628 | |
| | | | (albumin D-box) binding protein | | |
| 19 | J03179_g_at | DBP | DBP: D site of albumin promoter | LocusID: 1628 | |
| | | | (albumin D-box) binding protein | | |
| 20 | M31837_at | IGFBP3 | IGFBP3: insulin-like growth factor | LocusID: 3486 | |
| | | | binding protein 3 | | |
| 21 | M64711_at | EDN1 | EDN1: endothelin 1 | LocusID: 1906 | |
| 22 | M80367_at | 2633 | GBP1: guanylate binding protein | LocusID: 2633 | |
| | | | 1, interferon-inducible, 67kD | | |
| 23 | D89730_at | | | | |
| 24 | J03624_at | GAL | GAL: galanin | LocusID: 2586 | |
| 25 | U18314_g_at | 7112 | TMPO: thymopoietin | LocusID: 7112 | |
| 26 | U45965_at | | GRO3 ONCOGENE | LocusID: 2921 4q21 | |
| 27 | AF009329_at | DEC2 | DEC2: bHLH protein DEC2 | LocusID: 79365 | |
| 28 | M10934_s_at | RBP4 | RBP4: retinol-binding protein 4, | LocusID: 5950 | |
| | | | plasma | | |
| 29 | | | | | |
| 30 | Z22607_at | BMP4 | BMP4: bone morphogenetic | LocusID: 652 | |
| | | | protein 4 | | |
| 31 | X58830_at | BMP6 | BMP6: bone morphogenetic | LocusID: 654 | |
| | | | protein 6B | | |
| 32 | U92564_g_at | | *FINGER PROTEIN | LocusID: Rn 94188 5q34 | |
| 33 | D49494UTR#1_g_at | GDF10 | GDF10: growth differentiation | LocusID: 2662 | |
| | | | factor 10 | | |
| 34 | | | | | |
| 35 | M84719_at | FMO1 | FMO1: flavin containing | LocusID: 2326 | |
| | | | monooxygenase 1 | | |
| 36 | rc_AA859837_at | GDA | GDA: guanine deaminase | LocusID: 9615 | |
| 37 | M80633_at | | | | |
| 38 | rc_AI172247_at | XDH | XDH: xanthene dehydrogenase | LocusID: 7498 | |
| 39 | rc_AI232374_g_at | H1F0 | H1F0: H1 histone family, member | LocusID: 3005 | |
| | | | 0 | | |
| 40 | rc_AA965261_at | H2AFY | H2AFY: H2A histone family, | LocusID: 9555 | |
| | | | member Y | | |
| 41 | | | | | |
| 42 | X71127_at | C1QB | C1QB: complement component | LocusID: 713 | |
| | | | 1, q suboomponent, beta | | |
| | | | polypeptide | | |
| 43 | M92059_s_at | 1675 | DF: D component of complement | LocusID: 1675 | |
| | | | (adipsin) | | |
| 44 | L03201_at | CTSS | CTSS: cathepsin S | LocusID: 1520 | |
| 45 | | | | | |
| 46 | J03637_at | Aldh3a1 | Aldh3: Aldehyde dehydrogenase | LocusID: 25375 | |
| | | | class 3 | | |
| 47 | M15327_at | ADH6 | ADH6: alcohol dehydrogenase 6 | LocusID: 130 | |
| | | | (class V) | | |
| 48 | X72792cds_s_at | 126 | ADH1C: alcohol dehydrogenase | LocusID: 126 | |
| | | | 1C (class I), gamma polypeptide | | |
| 49 | M27434_s_at | | | | |
| 50 | X68101_at | | | | |
| 51 | rc_AA799666_at | | | | |
| 52 | rc_AA874875_at | | | | |
| 53 | rc_AA891069_at | 1198 | CLK3: CDC-like kinase 3 | LocusID: 1198 | |
| 54 | rc_AA893244_at | | | | |
| 55 | rc_AA894089_s_at | | | | |
| 56 | rc_AI639246_at | | | | |
| 57 | | | | | |
| 58 | | | | | |
| 59 | | | | | |
| 60 | | | | | |
| 61 | | | | | |
| 62 | | | | | |
| 63 | | | | | |
| 64 | | | | | |

*Rat gene no apparent Human Homologue

RAT GENES IN RESTENOSIS: 3 HOUR UP REGULATED GENES AND HUMAN HOMOLOGUES 3

| | A | B | C | D | |
|---|---|---|---|---|---|
| 1 | Rat Accession # | Locus link | DESCRIPTION | Human Locus | E |
| 2 | | | | | |
| 3 | AA848563_s_at | 3303 | HSPA1A: heat shock 70kD protein | LocusID: 3303 | |
| | | | 1A | | |
| 4 | Z27118cds_s_at | | | | |
| 5 | rc_AA818604_s_at | | HSPA1L: heat shock 70kD protein- | LocusID: 3305 | 14q24 1 |
| | | | like 1 | | |
| 6 | S74351_s_at | 1843 | DUSP1: dual specificity | LocusID: 1843 | |
| 7 | X68041cds_s_at | SOD3 | SOD3: superoxide dismutase 3, | LocusID: 6649 | |
| | | | extracellular | | |
| 8 | | | | | |
| 9 | | | | | |
| 10 | rc_AA800784_at | 3491 | CYR61: cysteine-rich, angiogenic | LocusID: 3491 | |
| | | | inducer, 61 | | |
| 11 | L05489_at | DTR | DTR: diphtheria toxin receptor | LocusID: 1839 | |
| | | | (heparin-binding epidermal growth | | |
| | | | factor-like growth factor) | | |
| 12 | rc_AA858520_at | Fst | *Fst: Follistatin | LocusID: 24373 | MGC14128 |
| | | | | | hypothetical protein |
| | | | | | MGC 14128; LocusID |
| | | | | | 84985 |
| 13 | rc_AA858520_g_at | Fst | *Fst: Follistatin | LocusID: 24373 | |
| 14 | M19651_at | FOSL1 | FOSL1 FOS-like antigen-1 | LocusID: 8061 | |
| 15 | U19866_at | | | | |
| 16 | X13167cds_s_at | 4774 | NFIA: nuclear factor I/A | LocusID: 4774 | |
| 17 | Z54212_at | EMP1 | EMP1: epithelial membrane protein | LocusID: 2012 | |
| | | | 1 | | |
| 18 | | | | | |
| 19 | | | | | |
| 20 | M61875_s_at | | | | |
| 21 | AB015432_s_at | 8140 | SLC7A5: solute carrier family 7 | LocusID: 8140 | |
| | | | (cationic amino acid transporter, y+ | | |
| | | | system), member 5 | | |
| 22 | | | | | |
| 23 | | | | | |
| 24 | AF004811_at | MSN | MSN: moesin | LocusID: 4478 | |
| 25 | AF028784mRNA#1_s_at | GFAP | GFAP: glial fibrillary acidic protein | LocusID: 2670 | |
| 26 | AJ002556_s_at | 29457 | *Mtap6: microtubule-associated | LocusID: 29457 | |
| | | | protein 6 | | |
| 27 | rc_AA799773_at | 2318 | FLNC: filamin C, gamma (actin- | LocusID: 2318 | |
| | | | binding protein-280) | | |
| 28 | rc_AA799773_g_at | 2318 | FLNC: filamin C, gamma (actin- | LocusID: 2318 | |
| | | | binding protein-280) | | |
| 29 | | | | | |
| 30 | | | | | |
| 31 | rc_A1029183_s_at | 2697 | GJA1: gap junction protein, alpha 1, | LocusID: 2697 | |
| | | | 43kD (connexin 43) | | |
| 32 | L12407_at | DBH | DBH: dopamine beta-hydroxylase | LocusID: 1621 | |
| | | | (dopamine beta-monooxygenase) | | |
| 33 | M74494_g_at | Atp1a1 | Atp1a1: ATPase, Na+K+ | LocusID: 24211 | |
| | | | transporting, alpha 1 polypeptide | | |
| 34 | | | | | |
| 35 | | | | | |
| 36 | X13722_at | | | | |
| 37 | | | | | |
| 38 | | | | | |
| 39 | X71898_at | PLAUR | PLAUR: plasminogen activator, | LocusID: 5329 | |
| | | | urokinase receptor | | |
| 40 | | | | | |
| 41 | | | | | |
| 42 | rc_AA892813_s_at | MBLL | MBLL: C3H-type zinc finger protein, | LocusID: 10150 | |
| | | | similar to D melanogaster | | |
| | | | muscleblind B protein | | |
| 43 | rc_AA894318_at | | | | |
| 44 | S56464mRNA_g_at | | | | |
| 45 | X74S6Scds_at | PTB | PTB: polypyrimidine tract binding | LocusID: 5725 | |
| | | | protein (heterogeneous nuclear | | |
| | | | ribonucleoprotein I) | | |
| 46 | X62875mRNA_g_at | | | | |
| 47 | AF015953_at | 406 | ARNTL: aryl hydrocarbon receptor | LocusID: 406 | |
| | | | nuclear translocator-like | | |
| 49 | rc_AI639343_at | | | | |
| 50 | rc_H31747_s_at | | | | |
| 51 | rc_AA800708_at | | | | |
| 52 | rc_AA875405_at | | | | |
| 55 | | | | | |
| 56 | | | | | |
| 58 | | | | | |

*Rat gene no apparent Human Holomogue

RAT GENES IN RESTENOSIS: 3 DAY DOWN REGULATED AND HUMAN HOMOLOGUES

| 1 | A | | | | E Locus | F | G |
|---|---|---|---|---|---|---|---|
| 2 | RAT ACCESSION NO | B | C | D | Link | DESCRIPTION | HUMAN LOCUS |
| 3 | M17526_g_at | RR | | 2775 | 50664 | RATBPGTPC: GTP binding | LocusID: 50664 |
| | | | | | | protein | |
| 4 | M60921_at | 601597 | | | BTG2 | BTG2: BTG family, member 2 | LocusID: 7832 |
| 5 | M60921_g_at | | | | | | |
| 6 | rc_AA944156_s_at | 601597 | | | | GENE2: BTG2 | Gene map locus 1q32 |
| 7 | rc_AA925717_at | 605276 | Reddy E P | | 9625 | AATK: apoptosis-associated | LocusID: 9625 |
| 8 | M62752_at | Wang E | Leffers H | 602959 | 24799 | Stnl: Stalin-like protein**RAT | LocusID: 24799 |
| 9 | rc_H31144_g_at | PMID, | Greene | http:/www.n | | | |
| | | 8497321 | | cbi.nlm.nih.g | | | |
| | | | | ov/LocusLin | | | |
| | | | | k/LocRpt.cgi | | | |
| | | | | ?l=10188 | | | |
| 10 | rC_AI103238_at | | 604325 | | | PROTEIN PHOSPHATASE 2, | LocusID: 5521, Gene |
| | | | | | | REGULATORY SUBUNIT B, | map locus 5q31-33 |
| | | | | | | BETA, PPP2R2B | |
| 11 | Y13275_at | Zoller M | | | 7103 | TM4SF3: transmembrane 4 | LocusID: 7103 |
| 12 | | | | | | | |
| 13 | | | | | | | |
| 14 | rc_AI71462_s_at | | | 600074 | | CD24: ANTIGEN, CD24 | LocusID: 934, Gene |
| | | | | | | | map locus 6q21 |
| 15 | U49062_at | Hirokawa K | Y | | | CD24: ANTIGEN, CD24 | |
| 16 | U49062_g_at | | Altevogt P | | | | |
| 17 | M63282_at | Taub R | S | | | none: rat leucine zipper | |
| 18 | X58830_at | BMP6 | Wooley D E | 112266 | | BMP6: bone morphogenetic | LocusID: 654 |
| | | | | | | protein 6 | |
| 19 | rc_H31479_at | | | | | | |
| 20 | rc_AA859882_s_at | | | | | | |
| 21 | | | | | | | |
| 22 | | | | | | | |
| 23 | L00088expanded_cds#2_at | | | | | | |
| 24 | rc_AI639532_at | Leiden J M | | | | | |
| 25 | rc_AI136540_at | Ginard B | | | | | |
| 26 | M73701_at | | 191043 | | | SKELETAL MUSCLE | LocusID: 7136 Gene |
| | | | | | | | map locus 11p15 5 |
| 27 | rc_AI639096_at | NM 004533 | | | | | |
| 28 | X00975_at | Levy Z | | | | | |
| 29 | X00975_at | Levy Z | | | | | |
| 30 | X02412_at | Ginard B | | | | | |
| 31 | S68736_at | Rotkind M | | | | | |
| | | Lanfranchi | | | | | |
| 32 | rc_AA799471_at | G | | | | | |
| 33 | rc_AA851223_at | 172430 | | | | Enolase 1 | LocusID: 2023, Gene |
| | | | | | | | map locus 1pter- |
| | | | | | | | p36.13 |
| 34 | M75148_at | | | | | | |
| 35 | M10140_at | | | 123310 | | CREATINE KINASE, | LocusID: 1158, Gene |
| | | | | | | MUSCLE TYPE, CKM | map locus 19q13 |
| 36 | L24897_s_at | Ryan A F | | | | | |
| 37 | L10669_g_at | | 232600 | | | GLYCOGEN STORAGE | LocustID: 5837, Gene |
| | | | | | | DISEASE V | map locus 11q12- |
| | | | | | | | q13 2 |
| 38 | L10669_at | | | | | | |
| 39 | K02423cds_s_at | | | | | | |
| 40 | J00692_at | Nedel U. | | | | | |
| 41 | M63656_s_at | | 103870 | | | ALDOLASE C, FRUCTOSE- | LocusID: 230, Gene |
| | | | | | | BISPHOSPHATE, ALDOC | map locus 17cen-q12 |
| 42 | | | | | | | |
| 43 | | | | | | | |
| 44 | U14398_at | TC | 600103 | | | SYNAPTOTAGMIN 4 | LocusID: 6860, Gene |
| | | | | | | | map locus 18q12 3 |
| 45 | U14398_g_at | | | | | | |
| 46 | U16802_at | 604667 | 9289400 | | | Ca2+ de[emdemt actovator | LocustID: 8616, Gene |
| | | | | | | protein for secretion, CADPS | map locus 3p23-cen |
| 47 | rc_AI175539_at | 168890 | | | | PARVALBUMIN, PVALB | LocusID: 5816, Gene |
| | | | | | | | map locus 22q12- |
| | | | | | | | q13 t |
| 48 | M99223_at | | | | | | |
| 49 | M26161_at | North R A | | | | | |
| 50 | AF055477_at | D | | | | | |
| 51 | rc_AI177026 at | 182310 | JX | | 477 | ATP1A2: ATPase, Na+/K+ | LocustID: 477 |
| | | | | | | transporting, alpha 2 (+) | |
| | | | | | | polypeptide | |
| 52 | | | | | | | |
| 53 | | | | | | | |
| 54 | A04674cds_s_at | | 113730 | | | UNCOUPLING PROTEIN 1: | LocusID: 7350, Gene |
| | | | | | | UCP1 | map locus 4q31 |
| 55 | rc_AI044900_s_at | B | A | | | | |
| 56 | L46791_at | Grogan | | | | | |
| | | WM | | | | | |
| 57 | D43623_g_at | | Terada H. | | | | |
| 58 | J02585_at | | | | | | |
| 59 | | | | | | | |
| 60 | | | | | | | |
| 61 | AF019974_at | Metz | | | | | |
| 62 | AF031878_at | Parysek L M | | | | | |
| 63 | M85214_at | Matsuda I | | | | | |
| 64 | U17254_g_at | Milbrandt J | Olsson T | | | | |
| 65 | U88958_at | | Theill L E | | | | |
| 66 | L21192_at | | 162060 | | | GROWTH ASSOCIATED | LocusID: 2596, Gene |
| | | | | | | PROTEIN 43, GAP43 | map locus 3q13 1- |
| | | | | | | | q13 2 |
| 67 | AF031880_at | Saarma M | | | | | |
| 68 | | | | | | | |
| 69 | | | | | | | |
| 70 | X79321_at | | | | | | |
| 71 | K03242_at | *159440 | | | | MYELIN PROTEIN ZERO, | LocusID: 4359, Gene |
| | | | | | | MPZ | map locus 1q22 |
| 72 | M73049_at | | 605336 | | | INTERNEXIN, ALPHA, INA | LocusID: 9118, 10cen- |
| | | | | | | | q26 11 |
| 73 | X52817cds_s_at | | | | | | |
| 74 | X90475cds_at | | | | | | |
| 75 | D10699_at | | | | | | |
| 76 | D10699_g_at | HL | 191342 | | | UBIQUITIN CARBOXYL- | LocusID: 7345, Gene |
| | | | | | | TERMINAL ESTERASE L1, | map locus 4p14 |
| | | | | | | UCHL1 | |
| 77 | rc_AA957930_s_at | | | | | | |
| 78 | rc_AI227608_s_at | Ginzburg I | *157140 | | | MICROTUBULE-ASSOCIAT- | LocusID: 4137, Gene |
| | | | | | | ED PROTEIN TAU, MAPT | map locus 17q21 1 |
| 79 | rc_AI044508_s_{—l at} | FE | | | | | |
| 80 | U03414_s_at | Sutcliffe J G | Kihara I | | | | |
| 81 | Z12152_at | Brophy P J | PJ | | | | |
| 82 | X86789_at | | AM | | | | |
| 83 | rc_0AA875659_s_at | 605336 | | | | INTERNEXIN, ALPHA, INA, | LocusID: 9118 |
| | | | | | | NEUROFILAMENT PROTEIN | |
| | | | | | | 5 | |
| 84 | Z29649_at | Brophy P J | Lupski J R | | | | |
| 85 | M72711_at | G | | | | | |
| 86 | M24852_at | | | | | | |
| 87 | rc_AI072770_st | | | | | | |
| 88 | | | | | | | |
| 89 | | | | | | | |
| 90 | rc_AI171644_s_at | B | *602009 | | | CYTOCHROME c | LocusID: 1339 |
| | | | | | | OXIDASE, SUBUNIT VIa, | |
| | | | | | | POLYPEPTIDE 2, COX6A2 | |
| 91 | U78977_at | | | | | | |
| 92 | M37942exon#2-3_s | Holmes E W | | | | | |
| 93 | | | | | | | |
| 94 | | | | | | | |
| 95 | M27925_at | Greengard | | | | | |
| | | P | | | | | |
| 96 | rc_AI145494_g_at | | | | | | |
| 97 | rc_AI145494_at | P | | | | | |
| 98 | L25633_g_at | Eipper B A | Eipper B A | | | | |
| 99 | L31621_s_at | Patrick J | | | | | |
| 100 | U17604_at | Seki N | *600865 | | | RETICULON 1, RTN1 | LocusID: 6252, Gene |
| | | | | | | | map locus 4q21-22 |
| 101 | S50879_g_at | | | | | | |
| 102 | rc_AI043796_s_at | 193001 | | | | SLC18A2 | LocusID: 6571, Gene |
| | | | | | | | map locus 10q25 |
| 103 | U02983_at | | | | | | |
| 104 | X06655 at | 313475 | TC | | | SYNAPTOPHYSIN, SYP | LocusID: 6855, Gene |
| | | | | | | | map locus Xp11 23- |
| | | | | | | | p11 22 |
| 105 | L00603_at | Hoffman | | | | | |
| | | B J, Sabban | | | | | |
| 106 | L12407_at | E L | | | | | |
| 107 | U25967_at | | | 602753 | | ARISTALESS HOMEO BOX, | LocusID: 401, Gene |
| | | | | | | DROSOPHILA, HOMOLOG | map locus 11q13 3- |
| | | | | | | OF ARIX | q13 4 |
| 108 | M93669_at | Neill J D | | | | | |
| 109 | | | | | | | |
| 110 | | | | | | | |
| 111 | rc_AI639444_g_at | Ozanne B W | | | | | |
| 112 | X03369_s_at | 191130 | | | | TUBULIN, BETA, TUBB | LocusID: 7280, Gene |
| | | | | | | | map locus 6p21 3 |
| 113 | X59149_at | CH | | | | | |
| 114 | U30938_at | 157130 | | | | MICROTUBULE-ASSOCIAT- | LocusID: 4133; Gene |
| | | | | | | PROTEIN 2; ED MAP2 | map locus: 2q34-q35 |
| 115 | | | | | | | |
| 116 | | | | | | | |
| 117 | U32314_g_at | 266150 | | | | PYRUVATE CARBOXYLASE | LocusID: 5091, Gene |
| | | | | | | DEFICIENCY | map locus 11q13 4- |
| | | | | | | | q13 5 |
| 118 | M27434_s_at | | D | | | | |
| 119 | K01934mRNA#2_at | HC | | | | | |
| 120 | M10244_at | | 191290 | | | TYROSINE HYDROXYLASE, | LocusID: 7054, Gene |
| | | | | | | TH | map locus 11p15 5 |
| 121 | M15327_atat | | | | | | |
| 122 | | | | | | | |
| 123 | rc_AA799621_at | | | | | | |
| 124 | rc_AA799666_at | | | | | | |
| 125 | rc_AA892798_at | | | | | | |
| 126 | rc_AA893984_at | | | | | | |
| 127 | rc_AI638986_s_at | | | | | | |
| 128 | rc_AI639294_at | | | | | | |
| 129 | rc_H31550_at | JD | | | 64506 | CPEB1 cytoplasmic | LocusID: 64506 |
| | | | | | | polyadenytation element binding | |
| | | | | | | protein | |

RAT GENES IN RESTENOSIS: 3 DAY UP REGULATED GENES AND HUMAN HOMOLOGUES

| | A | | C | | E | F |
|---|---|---|---|---|---|---|
| 1 | RAT ACCESSION NO. | B | LINK | D | DESCRIPTION | HUMAN LOCUS |
| 2 | | | | | | |
| 3 | | | | | | |
| 4 | M36151cds_i_at | | 604305 | | MAJOR HISTOCOMPATIBILITY | LocusID: 3119, |
| | | | | | COMPLEX, CLASS II, DQ BETA-1; HLA- | Gene map locus: |
| | | | | | DQB1 | 6p21 3 |
| 5 | M15562_at | | | | | |
| 6 | rc_AI171966_at | | | | | |
| 7 | X14254cds_g_at | | P. | B. | | |
| 8 | X73371_at | Pecht I. | | | | |
| 9 | M32062_at | DW | 146790 | | Fc FRAGMENT OF IgG, LOW AFFINITY | LocusID: 2122; |
| | | | | | IIa, RECEPTOR FOR; FCGR2A | Gene map, locus: |
| | | | | | | 1q21-q23 |
| 10 | M10072mRNA_s_at | M. | | 151460 | PROTEIN-TYROSINE PHOSPHATASE, | LocusID: 5788; |
| | | | | | RECEPTOR-TYPE, C; PTPRC | Gene map locus: |
| | | | | | | 1q31-q32 |
| 11 | | | | | | |
| 12 | | | | | | |
| 13 | M98049_s_at | C. | 167805 | Verrando | PROLIFERATING CELL NUCLEAR | Gene map locus: |
| | | | | P | ANTIGEN; PCNA/PANCREATITIS- | 20p12/LocusID: |
| | | | | | ASSOCIATED PROTEIN; PAP | 5068; Gene map |
| | | | | | | locus: 2-12 |
| 14 | J05495_at | | T. | | | |
| 15 | U82612cds_at | 135600 | 2335 | | FIBRONECTIN 1; FN1 | LocusID: 2335; |
| | | | | | | Gene map locus: |
| | | | | | | 2q34 |
| 16 | U82612cds_g_at | 135600 | | | FIBRONECTIN 1; FN1 | LocusID: 2335; |
| | | | | | | Gene map locus: |
| | | | | | | 2q34 |
| 19 | AF100470_g_at | B. | M. | | | |
| 20 | J02722cds_at | S. | S. | | | |
| 21 | rc_AI179610_at | Koizumi | PA. | | | |
| | | S. | | | | |
| 22 | | | | | | |
| 23 | | | | | | |
| 24 | M24604_g_at | VJ. | *17674 | | PROLIFERATING CELL NUCLEAR | LocusID: 5111; |
| | | | 0 | | ANTIGEN; PCNA | Gene map locus: |
| | | | | | | 20p12 |
| 25 | U10894_s_at | SA. | 605356 | CJ. | TYROSINE 3- | LocusID: 7532; |
| | | | | | MONOOXYGENASE/TRYPTOPHAN 5- | Gene map locus: |
| | | | | | MONOOXYGENASE ACTIVATION | 7q11.23 |
| | | | | | PROTEIN, GAMMA ISOFORM; YWHAG | |
| 26 | rc_AA998164_s_at | 123836 | RJ | | CYCLIN B1; CCNB1 | LocusID: 891; Gene |
| | | | | | | map locus: 5q12 |
| 27 | U28938_at | 176884 | N. | 50677 | PROTEIN-TYROSINE PHOSPHATASE, | LocusID: 5786; |
| | | | | | RECEPTOR-TYPE, ALPHA; PTPRA | Gene map locus: |
| | | | | | | 20p13 |
| 28 | X17053mRNA_s_at | JR | Charo | | | |
| | | | I F. | | | |
| 29 | AB010119_at | Zhang | 42199 | MS. | DIc90F: Dynein light chain 90F | LocusID: 42199 |
| | | M | | | *Drosophila | |
| 30 | rc_AI233219_at | 601521 | Tonnel | | ENDOTHELIAL CELL-SPECIFIC | LocusID: 11082 |
| | | | A B. | | MOLECULE 1; ESM1 | |
| 31 | rc_AA858520_g_at | Vries | 136470 | | FOLLISTATIN; FST | LocusID: 10468; |
| | | C J. | | | | Gene map locus; |
| | | | | | | 5q11.2 |
| 32 | | | | | | |
| 33 | | | | | | |
| 34 | U31866_g_at | | | | | |
| 35 | J02720_at | | 207800 | | ARGINASE | map locus: 6q23 |
| 36 | | | | | | |
| 37 | rc_AA860039_s_at | | | | | |
| 38 | rc_AA866443_at | | | | | |
| 39 | rc_AA891255_at | | | | | |
| 40 | rc_AA894029_at | | | | | |

RAT GENES IN RESTENOSIS: 7 DAY UP REGULATED GENES AND HUMAN HOMOLOGUES

| | A | | C | | E | F |
|---|---|---|---|---|---|---|
| 1 | RAT ACCESSION NO. | B | LINKS | D | DESCRIPTION | HUMAN LOCUS |
| 2 | | | | | | |
| 3 | M36151cds_at | | | | | |
| 4 | X14254cds_at | | | | | |
| 5 | U65217_i_at | | | | | |
| 6 | rc_AI171966 at | HLA-DMB | | | HLA-DMB: major | LocusID: 3109 |
| | | | | | histocompatibility complex, | |
| | | | | | class II, DM beta | |
| 7 | M61875_s_at | | | | | |
| 8 | J02962_at | | | | | |
| 9 | rc_AA859954_at | | | | | |
| 10 | | | | | | |
| 11 | | | | | | |
| 12 | M80829_at | TNNT2 | | | TNNT2: troponin T2, cardiac | LocusID: 7139 |
| 13 | AJ005394_at | 1289 | | | COL5A1: collagen, type V, | LocusID: 1289 |
| | | | | | alpha 1 | |
| 14 | AJ005396_at | | | | | |
| 15 | M12098_s_at | | | | | |
| 16 | L13606_at | | | | | |
| 17 | X51531cds_at | | | | | |
| 18 | X51531cds_g_at | | | | | |
| 19 | rc_AI104561_g_at | ACTC | | | ACTC: actin, alpha, cardiac | LocusID: 70 |
| | | | | | muscle | |
| 20 | rc_AA866452_s_at | ACTC | | | ACTC: actin, alpha, cardiac | LocusID: 70 |
| | | | | | muscle | |
| 21 | X80130cds_f_at | ACTC | *102540 | | | |
| 22 | X80130cds_i_at | | | | | |
| 23 | rc_AA800206_at | | | | | |
| 24 | rc_AI169327_g_at | TIMP1 | | | TIMP1: tissue inhibitor of | LocusID: 7076 |
| | | | | | metalloproteinase 1 (erythroid | |
| | | | | | potentiating activity, | |
| | | | | | collagenase inhibitor) | |
| 25 | X83537_at | MMP14 | | | MMP14: matrix | LocusID: 4323 |
| | | | | | metalloproteinase 14 | |
| | | | | | (membrane-inserted) | |
| 26 | X98517_at | MMP12 | | | MMP12: matrix | LocusID: 4321 |
| | | | | | metalloproteinase 12 | |
| | | | | | (macrophage elastase) | |
| 27 | U57362_at | COL12A1 | 120320 | | COL12A1: collagen, type XII, | LocusID: 1303 |
| | | | | | alpha 1 | |
| 28 | M14656_at | SPP1 | | | SPP1: secreted | LocusID: 6696 |
| | | | | | phosphoprotein 1 | |
| | | | | | (osteopontin, bone | |
| | | | | | sialoprotein I, early T- | |
| | | | | | lymphocyte activation 1) | |
| 29 | M88469_at | SPON1 | T. | | SPON1: spondin 1, (f- | LocusID: 10418 |
| | | | | | spondin) extracellular matrix | |
| | | | | | protein | |
| 30 | rc_AA859740_at | HS6ST | | | HS6ST: heparan sulfate 6-O- | LocusID: 9394 |
| | | | | | sulfotransferase | |
| 31 | rc_AA859757_at | COL5A1 | | | COL5A1: collagen, type V, | LocusID: 1289 |
| | | | | | alpha 1 | |
| 32 | rc_AA859757_g_at | | | | | |
| 33 | rc_AI102814_at | LOX | | | LOX: lysyl oxidase | LocusID: 4015 |
| 34 | rc_AA875582_at | LOX | HM | | LOX: lysyl oxidase | LocusID: 4015 |
| 35 | rc_AA875047_at | CCT6A | | | | |
| 36 | rc_AA875665_at | | *603420 | | CALU: calumenin | LocusID: 813 |
| 37 | D90258_s_at | 5683 | | | PSMA2: proteasome | LocusID: 5683 |
| | | | | | (prosome, macropain) subunit, | |
| | | | | | alpha type, 2 | |
| 38 | | | | | | |
| 39 | | | | | | |
| 40 | S81497_s_at | | | | | |
| 41 | AB005900_at | OLR1 | | | OLR1: oxidised low density | LocusID: 4973 |
| | | | | | lipoprotein (lectin-like) | |
| | | | | | receptor 1 | |
| 42 | L07114_at | | | | | |
| 45 | L35271_at | RUNX1 | | | RUNX1: runt-related | LocusID: 861 |
| | | | | | transcription factor 1 (acute | |
| | | | | | myeloid leukemia 1, aml1 | |
| | | | | | oncogene) | |
| 46 | X59864mRNA_at | Verrell | | | | |
| | | ePl | | | | |
| 47 | rc_891041_at | JUNB | | | JUNB: jun B proto-oncogene | LocusID: 3726 |
| 48 | | | | | | |
| 49 | | | | | | |
| 50 | AF012891_at | SFRP4 | | | SFRP4: secreted frizzled- | LocusID: 6424 |
| | | | | | related protein 4 | |
| 51 | rc_AA866465_s_at | NTRK1 | #164970 | *1913 | NIRK1: neurotrophic tyrosine | LocusID: 4914 |
| | | | | 15 | kinase, receptor, type 1 | |
| 52 | D14076_at | 1759 | Hirokawa | Feron | DNM1: dynamin 1 | LocusID: 1759 |
| | | | N | O. | | |
| 53 | J03627_at | S100A10 | | | S100A10: S100 calcium- | LocusID: 6281 |
| | | | | | binding protein A10 (annexin II | |
| | | | | | ligand, calpactin I, tight | |
| | | | | | polypeptide (p11)) | |
| 54 | U23407_at | CRABP2 | | | CRABP2: cellular retinoic acid | LocusID: 1382 |
| | | | | | binding protein 2 | |
| 55 | rc_AI171796_at | CAPN6 | | | CAPN6: calpain 6 | LocusID: 827 |
| 56 | L38644_at | KPNB1 | 602738 | | KPNB1: karyopherin (importin) | LocusID: 3837 |
| | | | | | beta 1 | |
| 59 | D00753_at | CHD2 | | | CHD2: chromodomain | LocusID: 1106 |
| | | | | | helicase DNA binding protein | |
| | | | | | 2 | |
| 60 | rc_AA899854_at | TOP2A | | | TOP2A: topoisomerase (DNA) | LocusID: 7153 |
| | | | | | II alpha (170kD) | |
| 61 | M89646_at | | | | | |
| 62 | rc_AI103498_at | RPL5 | | | RPL5: ribosomal protein L5 | LocusID: 6125 |
| 63 | X6O767mRNA_s_at | CDC2 | | | CDC2: cell division cycle 2, | LocusID: 983 |
| | | | | | G1 to S and G2 to M | |
| 64 | rc_AI008852_at | EEF1A1 | | | EEF1A1: eukaryotic | LocusID: 1915 |
| | | | | | translation elongation factor 1 | |
| | | | | | alpha 1 | |
| 65 | rc_AA894059_at | STK18 | | | STK18: serine/threonine | LocusID: 10733 |
| | | | | | kinase 18 | |
| 66 | AF052695_at | CDC20 | | | CDC20: CDC20 (cell division | LocusID: 991 |
| | | | | | cycle 20, S. cerevisiae, | |
| | | | | | homolog) | |
| 67 | rc_AA859827_at | UMPK | | | UMPK: uridine | |
| | | | | | monophosphate kinase | |
| 68 | | | | | | |
| 69 | | | | | | |
| 70 | rc_AA945737_at | CXCR4 | | | CXCR4: chemokine (C-X-C | LocusID: 7852 |
| | | | | | motif), receptor 4 (fusin) | |
| 71 | X17012mRNA_s_at | | | | | |
| 72 | M15481_g_at | 3479 | 265850 | | IGF1: insulin-like growth factor | LocusID: 3479 |
| | | | | | 1 (somatomedin C) | |
| 73 | rc_AA858520_at | Fst | | | Fst: Foltistatin* rat only | LocusID: 24373 |
| 74 | M24393_at | MYOG | | | MYOG: myogenin (myogenic | LocusID: 4656 |
| | | | | | factor 4) | |
| 75 | U87983_at | HMMR | | | HMMR: hyaluronan-mediated | LocusID: 3161 |
| | | | | | motility receptor (RHAMM) | |
| 76 | rc_AA894092_at | OSF-2 | | | OSF-2: osteoblast specific | LocusID: 10631 |
| | | | | | factor 2 (fasciclin I-like) | |
| 77 | rc_A1233219_at | ESM1 | | | ESM1: endothelial cell-specific | LocusID: 11082 |
| | | | | | molecule 1 | |
| 78 | U50736_s_at | CARP | | | CARP: cardiac ankyrin repeat | LocusID: 27063 |
| | | | | | protein | |
| 79 | X17053mRNA_s_at | SCYA2 | Eb AJ | | SCYA2: small inducible | LocusID: 6347 |
| | | | | | cytokine A2 (monocyte | |
| | | | | | chemotactic protein 1, | |
| | | | | | homologous to mouse Sig-je) | |
| 80 | X89963_at | THBS4 | | | THBS4: thrombospondin 4 | LocusID: 7060 |
| 81 | X02002_at | | | | | |
| 82 | rc_AA874848_s_at | THY1 | | | THY1: Thy-1 cell surface | LocusID: 7070 |
| | | | | | antigen | |
| 83 | X63722cds_s_at | | | | | |
| 84 | M84488_at | | | | | |
| 85 | L00191cds#1_s_at | | | | | |
| 86 | U82612cds_at | | | | | |
| 87 | U82612cds_g_at | | | | | |
| 88 | rc_AA893846_at | 7143 | | | TNR: tenascin R (restrictin, | LocusID: 7143 |
| | | | | | janusin) | |
| 89 | U09361_s_at | | | | | |
| 90 | U09401_s_at | | | | | |
| 91 | U15550_at | | | | | |
| 92 | L20869_at | 5068 | | | PAP: pancreatitis-associated | LocusID: 5068 |
| | | | | | protein | |
| 94 | | | | | | |
| 95 | M23697_at | PLAT | | | PLAT: plasminogen activator, | LocusID: 5327 |
| | | | | | tissue | |
| 96 | M19647_i_at | KLK1 | | | KLK1: kallikrein 1, | LocusID: 3816 |
| | | | | | renal/pancreas/salivary | |
| 97 | X74832cds_at | CHRNA1 | | | CHRNA1: cholinergic | LocusID: 1134 |
| | | | | | receptor, nicotinic, alpha | |
| | | | | | polypeptide 1 (muscle) | |
| 98 | X74835cds_at | CHRND | | | CHRND: cholinergic receptor, | LocusID: 1144 |
| | | | | | nicotinic, delta polypeptide | |
| 99 | | | | | | |
| 100 | | | | | | |
| 101 | U33441mRNA_s_at | | | | | |
| 102 | M31032cds#1_s_at | | | | | |
| 103 | M31O32mRNA#2_at | | | | | |
| 104 | M177O3mRNA_s_at | | | | | |
| 105 | M33976_at | | | | | |
| 106 | rc_AA892775_at | LYZ | | | LYZ: lysozyme (renal | LocusID: 4069 |
| | | | | | amyloidosis) | |
| 107 | X12459_at | | | | | |
| 108 | D14441_at | | | | | |
| 109 | rc_AA859536_at | Basp1 | *605940 | | BASP1: brain abundant, | LocusID: 10409 |
| | | | | | membrane attached signal | |
| | | | | | protein 1. *human | |
| 110 | rc_AI176456_at | | 4489 | | MT1A: metallothionein 1A | LocusID: 4489 |
| | | | | | (functional) | |
| 111 | | | | | | |
| 112 | rc_AA860039_s_at | | | | | |
| 113 | rc_AA860057_at | | | | | |
| 114 | rc_AA866290_at | | | | | |
| 115 | rc_AA866443_at | | | | | |
| 116 | rc_AA893717_at | | | | | |

What is claimed is:

1. A method for the detection of restenosis in a mammal, comprising assaying the level of expression of at least three genes in a sample obtained from said mammal.

2. The method according to claim 1, wherein the presence of restenosis is indicated by increased expression of a set of genes in said sample, wherein said set comprises at least three genes, at least five genes, at least ten genes, or at least twenty genes.

3. The method according to claim 1, wherein the presence of restenosis is indicated by decreased expression of a set of genes in said sample, wherein said set comprises at least three genes, at least five genes, at least ten genes, or at least twenty genes.

4. The method according to claim 1, wherein the presence of restenosis is indicated by the altered expression of a set of genes in said sample, wherein said set comprises at least three genes, at least five genes, at least ten genes, at least twenty genes, or at least fifty genes.

5. The method according to claim 1, wherein said genes are selected from the group consisting of the genes listed in Table 1.

6. The method according to claim 1, wherein said sample comprises vascular tissue of said mammal.

7. The method according to claim 6, wherein said vascular tissue is vascular arterial tissue.

8. The method according to claim 6, wherein said vascular tissue is vascular venous tissue.

9. The method according to claim 2, wherein said increased expression is at least two fold higher, at least four fold higher, or at least ten fold higher than a reference level.

10. The method according to claim 3, wherein said decreased expression is at least one-half or at least one-tenth the reference level.

11. The method according to claim 4, wherein said altered expression, when increased, is at least two fold higher than a reference level of that gene and when decreased, is at least one-half the level of that gene when compared to a reference level.

12. The method according to claim 9, wherein said reference level is the level in healthy vascular tissue, or is determined from pre-stenotic levels.

13. The method according to claim 10, wherein said reference level is the level in healthy vascular tissue, or is determined from pre-stenotic levels.

14. The method according to claim 11, wherein said reference level is the level in healthy vascular tissue, or is determined from pre-stenotic levels.

15. The method according to claim 1, wherein the assay is carried out using a method selected from the group consisting of: genetic microarray analysis, quantitative PCR, and assay of the level of protein expression in a sample.

16. The method according to claim 15, wherein said assay method is assay of the level of protein expression in a sample, and wherein said proteins are soluble proteins.

17. The method according to claim 16, wherein said sample is blood.

18. The method according to claim 16, wherein said sample is lymph.

19. The method according to claim 16, wherein the level of protein expression is determined by ELISA.

20. A method of inhibiting restenosis comprising administering to a patient suffering from restenosis a composition that inhibits smooth muscle cell proliferation or neointimal hyperplasia and wherein said composition modifies expression of at least one gene listed in Table 1.

21. The method according to claim 20, wherein said composition induces the expression of a gene or gene transcript that ameliorates effects of restenosis.

22. The method according to claim 20, wherein said composition inhibits genes which promote smooth muscle cell proliferation or neointimal hyperplasia.

23. The method according to claim 20, wherein said composition comprises a compound selected from the group consisting of an antisense oligonucleotide, an oligonucleotide that binds to mRNA to form a triplex, and an RNAi molecule.

24. The method according to claim 20, wherein said composition inhibits the activity of at least one protein that promotes smooth muscle cell proliferation or neointimal hyperplasia.

25. The method according to claim 24, wherein said composition comprises a composition selected from the group consisting of an antibody that binds to a protein that promotes smooth muscle cell proliferation or neointimal hyperplasia, and a soluble receptor protein.

26. The method according to claim 25, wherein said composition comprises a human antibody.

27. The method according to claim 20, wherein said composition comprises a protein that is administered to supplement the loss of a protein down-regulated during the course of restenosis.

28. The method according to claim 1, wherein detection is carried out using a kit suitable for performing PCR and wherein said kit comprises primers specific for the amplification of DNA or RNA sequences identified by the genes in Table 1.

29. A method of estimating the risk of developing restenosis or of atherosclerosis in an individual, comprising detecting the presence of biologically important polymorphisms in a set of genes in a sample obtained from said individual, wherein said set comprises at least three genes, at least five genes, at least ten genes, or at least fifty genes.

30. The method according to claim 29, wherein said genes are selected from the group consisting of the genes listed in Table 1.

31. The method according to claim 29, wherein said sample comprises venous or arterial blood of said individual.

32. The method according to claim 29, wherein said sample comprises vascular tissue, vascular arterial tissue, lymph, or blood of said individual.

33. The method according to claim 29, wherein said polymorphisms are detected using at least one method selected from the group consisting of genetic microarray and quantitative PCR.

34. The method according to claim 29, wherein detection is carried out using a kit suitable for detecting biologically significant polymorphisms of the genes in Table 1.
